(19) United States
(12) Patent Application Publication
(10) Pub. No.: US 2008/0249618 A1
(43) Pub. Date: Oct. 9, 2008

(54) Repair of Incompetent Heart Valves by Interstitial Implantation of Space Occupying Materials or Devices

(75) Inventors: Rany Huynh, Charlestown, MA (US); Theodore Lamson, Pleasanton, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/837,706

(22) Filed: Aug. 13, 2007

Related U.S. Application Data
(60) Provisional application No. 60/910,767, filed on Apr. 9, 2007.

(51) Int. Cl. A61F 2/24

(52) U.S. Cl. 623/2.11

(57) ABSTRACT

Incompetency or regurgitation of a cardiac valve is treated by injecting a space occupying material(s) or implanting a space occupying device(s) at an interstitial location adjacent to the valve such that the space occupying material or device exerts pressure on the valve causing one or more leaflets of the valve to be favorably repositioned. The procedure may be performed by open thoracotomy, thorascopically or transluminally using a tissue penetrating catheter.

### RELATED APPLICATIONS

This application claims the benefit of U.S. Provisional Patent Application 60/910,767 filed Apr. 9, 2007.

### FIELD OF THE INVENTION

The present invention relates generally to medical devices and methods, and more particularly to devices and methods for implanting space occupying materials or devices within cardiac tissue so as to exert pressure on the valve annulus thereby improving coaptation of the valve leaflets.

### BACKGROUND

In humans and many mammals, the heart includes a number of chambers and valves of located between the chambers of the heart to control the flow of blood from chamber to chamber.

For example, in humans, the mitral valve is located between the left atrium and left ventricle of the heart. The mitral valve consists of two leaflets (the anteromedial leaflet and the posterolateral leaflet) surrounded by a fibrous ring known as the mitral valve annulus. Two papillary muscles extend as finger-like projections from the wall of the left ventricle into the left ventricular cavity. Each papillary muscle is attached to a leaflets of the mitral valve by way of an inelastic tendon network known as the chordae tendineae. During the diastolic phase of the cardiac cycle, the left ventricular myocardium relaxes, thus causing the pressure within the left ventricle to decrease and causing the mitral valve leaflets to open as blood travels from the left atrium into the left ventricle. Thereafter, during the systolic phase of the cardiac cycle, the left ventricle contracts, thereby causing an increase in pressure within the left ventricle. This increase in left ventricular pressure causes the mitral valve leaflets to close, while their connection to the papillary muscles (via the chordae tendineae) prevents the mitral valve leaflets from prolapsing through the valve annulus in the wrong direction.

Mitral valve regurgitation (also known as mitral insufficiency or mitral incompetence) results when the leaflets of the mitral valve don't fully coapt (i.e., don't close tightly), thus allowing blood to backflow from the left ventricle into the left atrium during the systolic phase of the cardiac cycle. Mitral regurgitation can result from a number of causes. However, irrespective of its underlying etiology, mitral regurgitation can result in decreased cardiac output and inadequate perfusion of tissues throughout the body, with various resultant symptoms, including severe fatigue and shortness of breath.

The appropriate treatment for mitral valve regurgitation varies from case to case, depending on the severity and progression of the condition and age/condition of the patient. In severe cases, repair or replacement of the mitral valve may be indicated.

In recent years, a number of minimally invasive catheter-based procedures have been proposed for repairing regurgitating mitral valves without requiring open chest surgery. In some of these minimally invasive catheter-based procedures, a catheter device is advanced into a chamber of the heart (i.e., the left atrium or left ventricle) and is used to modify or constrain the mitral valve annulus, mitral valve leaflets and/or subvalvular apparatus (e.g., the papillary muscles and/or chordae tendineae) in a manner which ostensibly improves closure of the mitral valve leaflets. For example, U.S. Pat. No. 6,629,534 (St. Goar, et al.) describes methods, devices, and systems for the endovascular repair of cardiac valves (particularly the atrioventricular valves and most particularly the mitral valve) wherein interventional tools, catheters and other equipment are advanced though the vasculature and to the heart chambers. The interventional tools and other equipment are then used to modify the valve leaflets, the valve annulus, the chordae tendineae and/or the papillary muscles to improve closure of the mitral valve leaflets.

Also, United States Patent Application Publication No. 2007/0027533 describes a method and system wherein a catheter is advanced into the left atrium and used to deploy a restraining device that has barbs which engage the mitral valve annulus. An adjustment member is then used to adjust the radius of the restraining member thereby causing a corresponding change in the shape of the mitral valve annulus and resultant improvement in closure of the valve leaflets.

In other minimally invasive catheter-based procedures, a catheter is advanced into the coronary venous sinus or coronary vein adjacent to the mitral valve and such catheter is used to deploy an implantable device which then remains resident within the vasculature (e.g., within the coronary sinus or within the lumen of a coronary blood vessel) and exerts pressure on the mitral valve to improve coaptation of the mitral valve leaflets. For example, United States Patent Application Publication 2007/0010878 describes a method and procedure for treating mitral valve regurgitation wherein a catheter is used to implant a compression device within the coronary sinus adjacent to the mitral valve. The device implanted within the coronary sinus exerts a compressive force on the mitral valve for the purpose of causing the mitral valve leaflets to fully close during systole.

Also, United States Patent Application Publication No. 2006/0287968 describes devices and methods for treatment of mitral regurgitation by deployment of implantable devices within the anterior and posterior interventricular veins, or only in the posterior interventricular vein, to cause medial displacement of the anterior and posterior interventricular veins towards the left ventricular cavity. This in turn causes repositioning of the papillary muscles in a manner that purportedly brings the mitral valve leaflets into proper copatation during the systolic phase of the cardiac cycle.

There remains a need for the development of new devices and methods for the treatment of cardiac valve disorders such, as mitral valve regurgitation, without long term implantation of foreign objects within the chambers of the heart, coronary sinus or coronary vessels and without attachment of apparatus to the annulus or leaflets of the valve.

### SUMMARY OF THE INVENTION

The present invention provides methods and systems for modifying the function of a cardiac valve by placing an interstitial space occupier (e.g., a substance or device) at one or more location(s) within heart tissue near the valve such that the space occupier will alter the shape and/or function of the valve in a manner that provides a therapeutic benefit. The interstitial space occupier may be placed within the myocardium adjacent to the annulus of the heart valve to be treated such that it does not reside within or protrude into the coronary sinus or any coronary blood vessel lumen and, thus, does not obstruct or disrupt normal coronary blood flow. Also, the methods and systems of the present invention do not require attachment of any apparatus to the annulus or leaflets of the cardiac valve being treated.

In accordance with the present invention, there is provided a method for improving the function of an incompetent cardiac valve that has leaflets, such method comprising the step of implanting a space occupier (e.g., a substance or device) at an interstitial location within heart tissue such that force exerted by the space occupier causes repositioning of at least one of the valve leaflets to improve competency of the valve. In some instances, the space occupier may be delivered to the desired interstitial location by a tissue penetrating catheter device that has a delivery cannula (e.g., a hollow needle) that is advanceable and retractable from the catheter. The tissue penetrating catheter is advanced transluminally to a position within the coronary vasculature (e.g., the coronary sinus, coronary artery or coronary vein) and the delivery cannula is then advanced from the catheter, through the wall of the sinus or blood vessel in which the catheter is positioned, and to an interstitial location near the incompetent cardiac valve. The space occupier is then delivered through the delivery cannula, causing the space occupier to be implanted at an interstitial location within heart tissue such that force exerted by the space occupier causes repositioning of at least one of the valve leaflets to improve competency of the valve. In some embodiments, the space occupier may comprise an injectable filler substance such as collagen, hyaluronic acid, polymeric materials, hydrogels, etc. In other cases, the space occupier may comprise one or more device(s) such as beads or an expandable member in the nature of a stent or expandable cage.

Further aspects, elements, embodiments, objects and advantages of the present invention will be appreciated by those of skill in the relevant art upon reading the detailed description and examples set forth herebelow.

### BRIEF DESCRIPTION OF THE DRAWINGS

FIG. 1 is a sectional view of a human heart having a space occupier of the present invention implanted within cardiac tissue adjacent to the mitral valve for the treatment of mitral insufficiency.

FIGS. 2A-2F show a partial sectional view through the mitral valve of a human heart during the systolic phase of the cardiac cycle, with step-wise performance of one example of a method of the present invention for the treatment of mitral insufficiency.

FIG. 3 is schematic illustration showing a tissue penetrating catheter system operatively inserted into a human patient and being used to perform a cardiac valve treatment method of the present invention.

FIG. 3A is a side view of the tissue penetrating catheter device shown in FIG. 3.

FIG. 3B is an enlarged, partially fragmentary, elevational view of a distal portion of the tissue penetrating catheter device seen in FIG. 3A.

FIG. 3C is a non-fragmented cross sectional view through line 3C-3C of FIG. 3B.

FIG. 3D is a cross sectional view through line 3D-3D of FIG. 3B.

FIG. 3E is a cross sectional view through line 3E-3E of FIG. 3B.

FIG. 3F is a perspective view of the marker structure of the tissue penetrating catheter shown in FIGS. 3A-3E.

FIG. 3G is a non-fragmented cross sectional view through line 3G-3G of FIG. 3B.

FIG. 4A shows an example of an intravascular ultrasound image that the operator may see when the tissue penetrating catheter has been positioned within the coronary vasculature near the cardiac valve to be treated, but wherein the tissue penetrating catheter is not in the proper rotational orientation to cause its tissue penetrator to advance toward the intended interstitial location adjacent to the valve.

FIG. 4B shows an example of an intravascular ultrasound image that the operator may see when the tissue penetrating catheter has been positioned within the coronary vasculature near the cardiac valve to be treated and wherein the tissue penetrating catheter has been placed in the proper rotational orientation to cause its tissue penetrator to advance toward the intended interstitial location adjacent to the valve.

FIGS. 5A-5F show a partial sectional view through the mitral valve of a human heart during the systolic phase of the cardiac cycle, with step-wise performance of another example of a method of the present invention for the treatment of mitral insufficiency.

### DETAILED DESCRIPTION AND EXAMPLES

The following detailed description, the accompanying drawings are intended to describe some, but not necessarily all, examples or embodiments of the invention. The contents of this detailed description and accompanying drawings do not limit the scope of the invention in any way.

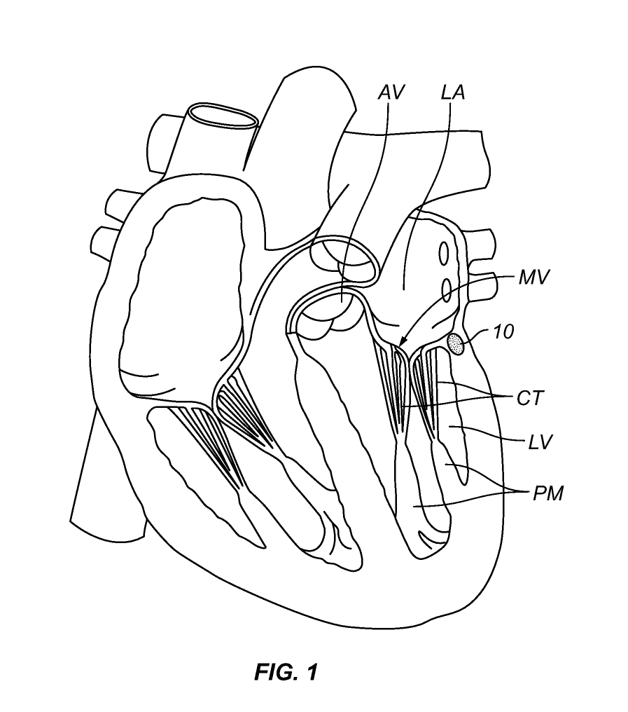

Referring to the accompanying drawings, FIG. 1 shows a sectional view of the heart of a human subject. The mitral valve MV is located between the left atrium LA and left ventrical LV, generally adjacent to the aortic valve AV. The papillary muscles PM are finger-like muscular projections that extend from the wall of the left ventricle, as shown. Inelastic tendons, known as the chordae tendineae CT extend from each papillary muscle PM to a leaflet of the mitral valve MV. In this example, a space occupier 10 (e.g., a quantity of a material or a device) has been implanted within the cardiac tissue, adjacent to the mitral valve MV. As explained fully herebelow, this space occupier 10 causes the position of a mitral valve leaflet to shift, thereby causing or improving coaptation of the leaflets during closure of the valve. This lessens mitral regurgitation and improves functioning of the mitral valve. At the same time, the space occupier 10 is wholly located within the cardiac tissue and does not protrude into or extend into the coronary sinus or the lumens of any coronary veins or arteries. Thus, the space occupier does not reside within, nor does it obstruct normal flow through, the coronary vasculature (e.g., the coronary sinus, coronary veins or coronary arteries).

In some embodiments, the space occupier 10 may comprise an injectable space occupying material 10a that forms at least one depot or mass at the interstitial location. In some cases, injection of the depot or mass may be in multiple locations around the annulus. The amount of material injected will be sufficient to exert pressure on the valve annulus, thereby causing the desired shift in the position of at least one valve leaflet and resulting in improved coaptation of the valve leaflets during closure of the valve. Non-limiting examples of injectable materials that may be used for this purpose include, but are not necessarily limited to, bulking agents, fat, collagens (e.g., collagens from human animal sources), crosslinked collagens (e.g., Zyplast®, Allergan-Inamed, Santa Barbara, Calif.), autologus collagen (Autologen; Collagenesis Inc., Beverly, Mass.); polymethylmethacrylate microspheres suspended in bovine collagen (Artecoll®; Rofil Medical International NV, Breda, The Netherlands), acellular freeze dried human cadaveric dermis (AlloDerm®, LifeCell Corporation, Branchburg, N.J.), micronized acellular freeze dried human cadaveric dermis (Cymetra®, LifeCell Corporation, Branchburg, N.J.), cultured autologous fibroblasts (Isolagen®, Isolagen Technologies, Inc., Exton, Pa.), hyaluronic acid, crosslinked hyaluronic acid (Hylaform® gel; Allergan-Inamed, Santa Barbara, Calif.; and Genzyme Corporation, Cambridge, Mass.), stabilized hyaluronic acid derivatives (Restylane®, Q-Med AB, Uppsala, Sweden), calcium hydroxyl appetite suspension (Radiesse®, Bioform Medical, Inc., San Mateo, Calif.), solubilized elastin peptides with bovine collagen (Endoplast-50®, Laboratories Filorga, Paris, France), dextran beads suspended in hylan gel (Reviderm®, Rofil Medical International NV, Breda, The Netherlands), silicones (e.g., high-viscosity liquid silicone such as Adatosil-5000™ and Silikon-1000™, Dow Corning, Midland Mich.), poly-L-lactic acid (Sculptra®, Dermik Aesthetics, Berwyn, Pa.), expanded polytetrafluoroethylene (e-PTFE) (e.g., SoftForm™ from Collagen Aesthetics, Inc./Allergan-Inamed, Santa Barbara, Calif. or Advanta™ from Atrium Medical Corporation, Hudson, N.H.), etc.

In other embodiments, the space occupier 10 may comprise one or more implantable space occupying device(s) 10b. In some cases a single space occupying device 10b may be used and in other cases, multiple devices 10b may also be place at locations around the annulus or in bunches around or near the annulus. Such implantable space occupying device(s) 10b may comprise one or more relatively simple space occupying articles or apparatus such as, for example, beads, balls, filament(s), strand(s), coils, suture material, etc. Or, such implantable device(s) may comprise and expandable implant such as a stent, an expandable cage, expandable cylinder, expandable ball, other expandable structures, implantable balloons, implantable balloons filled with solid or gellatenous material and implantable, tissue expanders, etc.

During injection of the space occupying material 10a or during implantation and/or expansion of the space occupying device 10b, the operator may observe the movement of the valve leaflets continually in real time, or at selected intervals, to determine when sufficient pressure is being applied to the valve to bring the leaflets into improved closure during the phase of the cardiac cycle when that particular valve should close (e.g., during systole in the case of a mitral valve).

In some application of the invention, the injectable material or device comprising the space occupier 10 may be injected or introduced into the desired interstitial location during an open-chest surgical procedure or using minimally invasive thoracoscopic techniques known in the art. In other applications, the injectable material or device comprising the space occupier 10 may be delivered by catheter(s) that are a) advanced through the subject's vasculature to a position within the coronary vasculature (e.g., in the coronary sinus, coronary vein or coronary artery), b) used to penetrate from the coronary vasculature to the desired interstitial location and c) used to deliver the material or device comprising the space occupier 10 to the interstitial location.

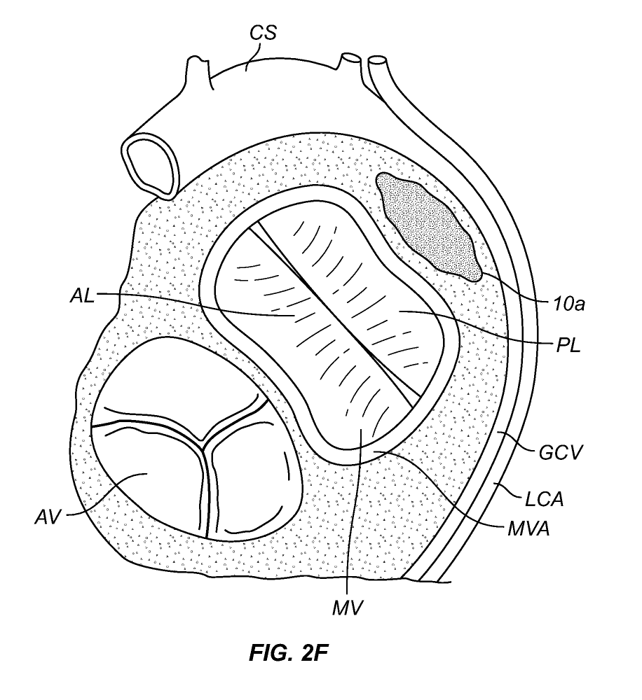

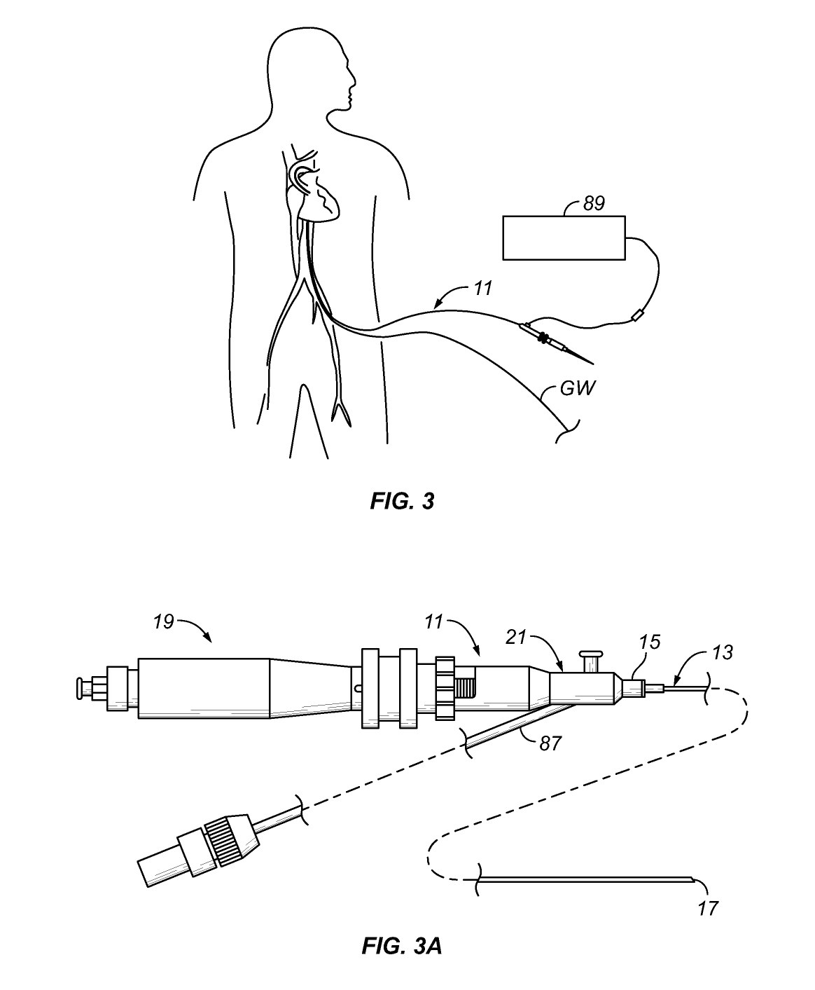

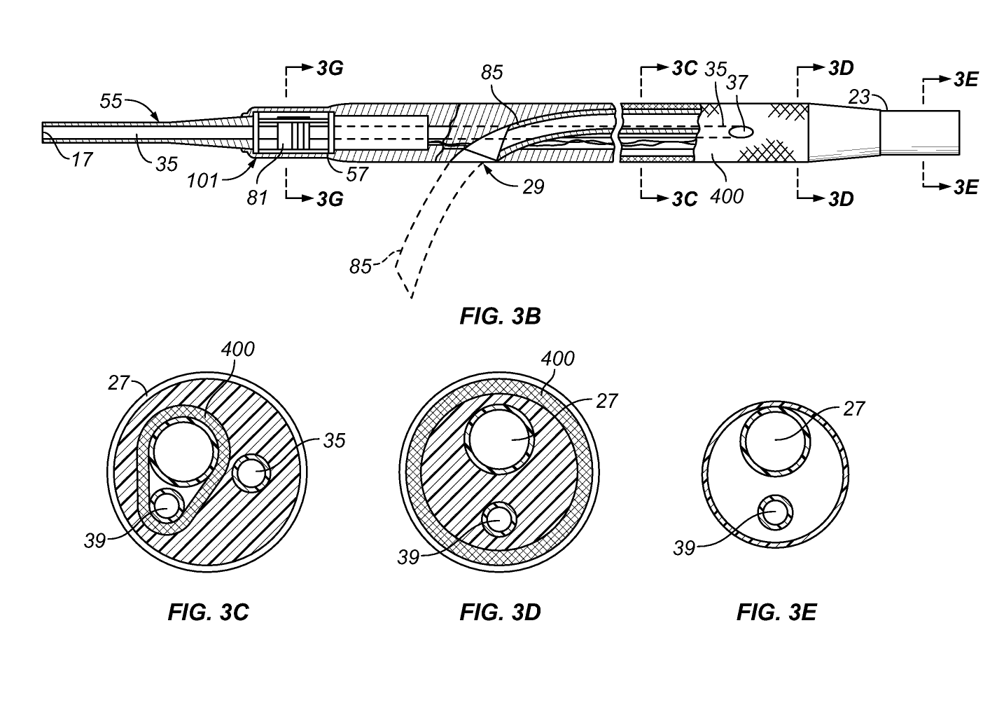

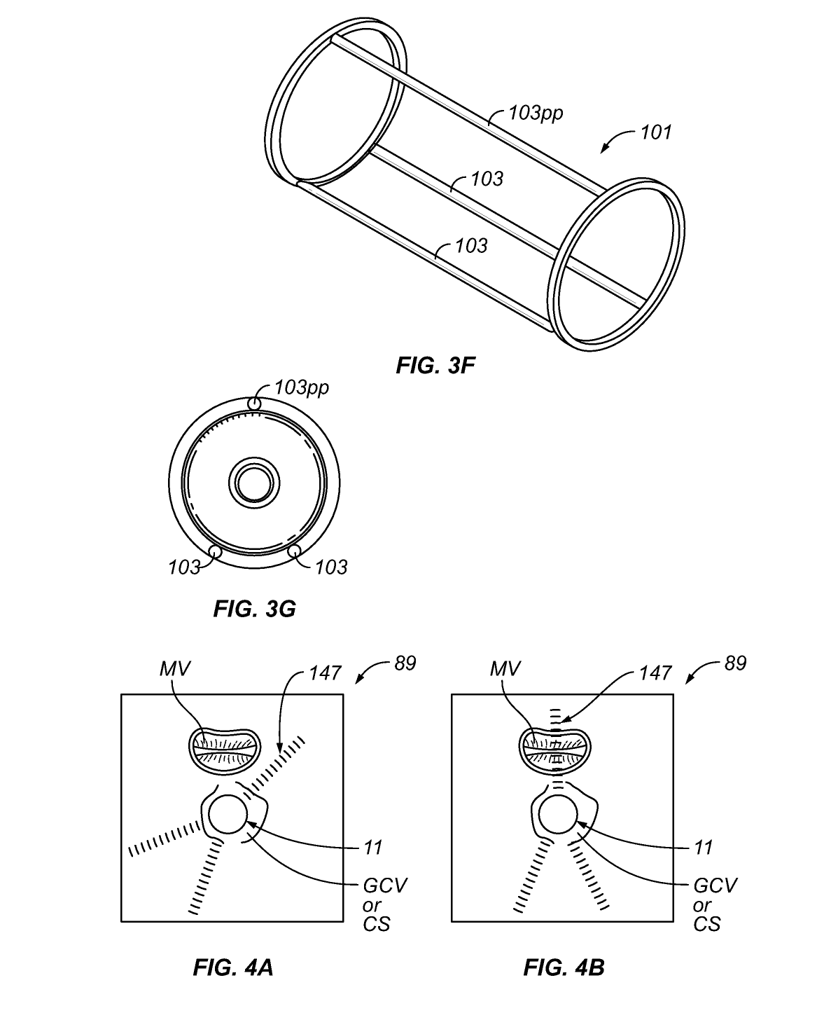

FIGS. 3A-3G show a tissue penetrating catheter 11 that may be used to perform the methods of the present invention. This catheter 11 includes an elongated catheter body 13 having a proximal end 15, a distal end 17, a handle 19 and a hub 21 coupled to the proximal end of the catheter body 15 and to the handle. The handle 19 may also serve as a controller for use in advancing and retracting the penetrating instrument, such as a tissue penetrator 85 described more fully below.

### The Catheter Body

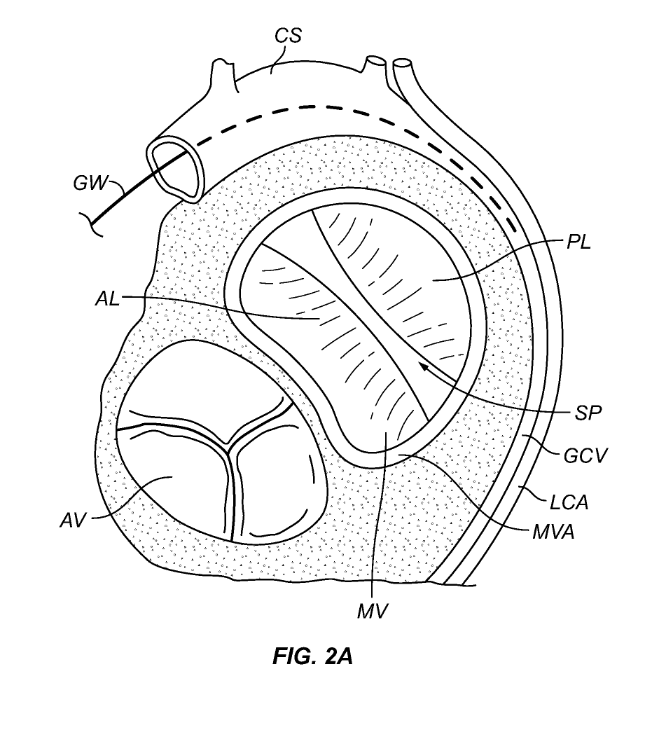

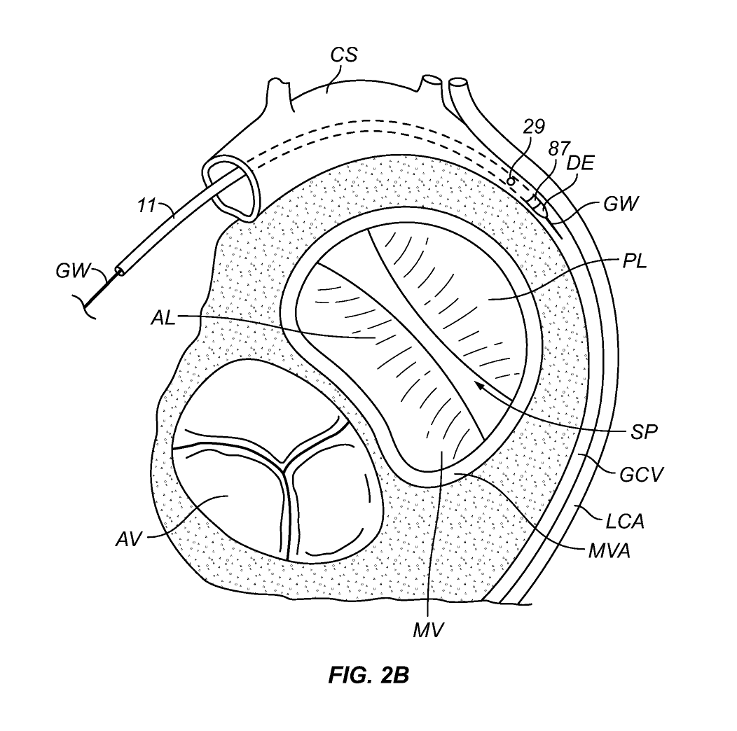

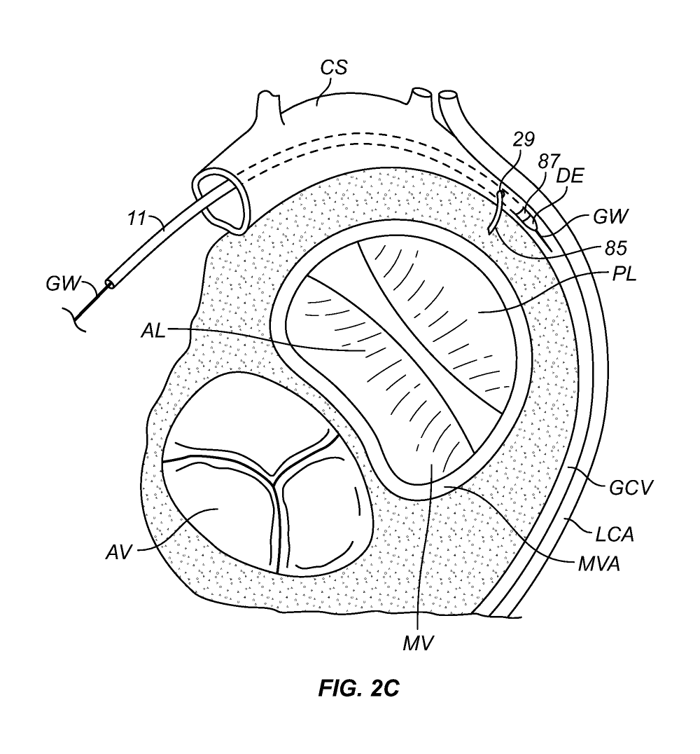

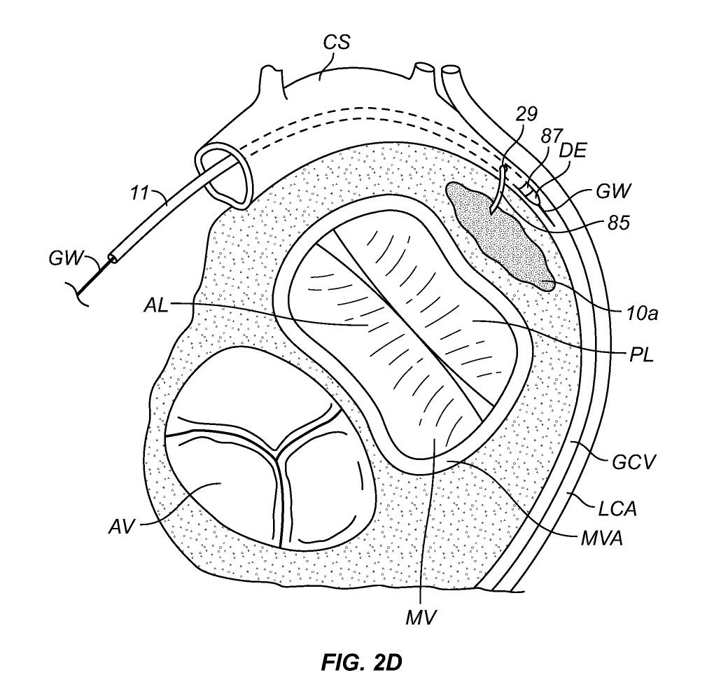

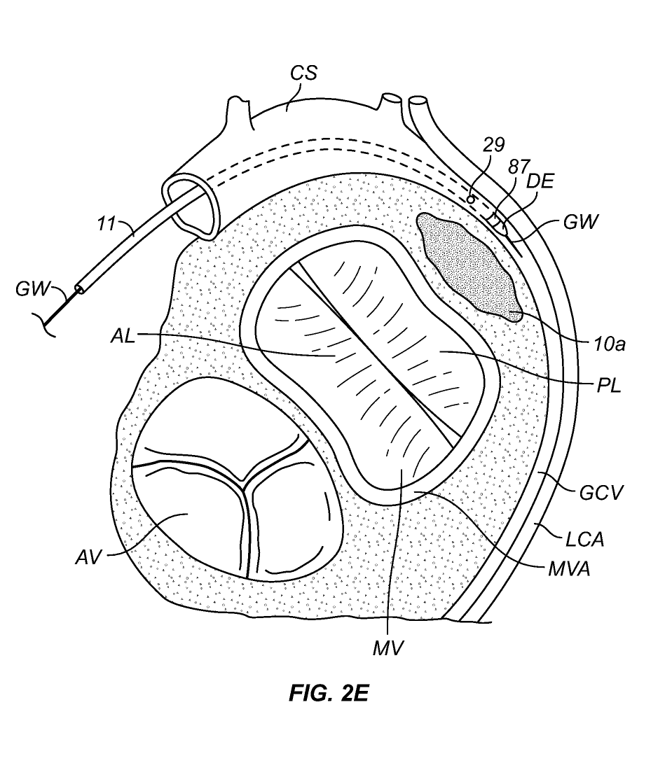

The catheter body 13 includes a relatively rigid proximal section 23 shown in FIGS. 2 and 3a which may be constructed, for example, of a metal hypo tube and an elongated flexible distal section or region suitably joined to and extending distally from the proximal section. A hand piece 19 is attached to the proximal end of the proximal section 23, as shown. In the preferred embodiment the hand piece 19 and proximal section 23 are approximately 100 cm in length. The flexible distal section may incorporate a reinforcement member such as a wire braid 400 as shown in FIG. 3D and, which in the example shown may be approximately 30 cm in length. This braid 400 may terminate approximately 3 cm from the distal end 17.

In this example, the catheter body 13 has a penetrator lumen 27 that terminates distally at an exit location or exit port 29 on the side wall of the catheter. The penetrator lumen 27 extends proximally from the exit port 29 to the proximal end 15 of the catheter body 13 and communicates with the interior of the handle 19 through the hub 21. The penetrator lumen 27 contains the tissue penetrator 85, which is advanceable from the catheter body 13 through the wall of the coronary sinus or coronary blood vessel in which the catheter body 13 is positioned and to an interstitial location within heart tissue. The exit port 29 is preferably located a short distance proximal to the distal tip 17. A radiopaque marker may be mounted on the lumen 27 adjacent the exit port 29.

In some applications, the space occupying substance may be formed by mixing two or more component substances. In such applications, an injector device having 2 or more lumens may be used to inject the component substances so that they become combined in situ at the implantation site or within the injection device shortly before the resultant component mixture enters the implantation site. Examples of multiple-component injector devices that may be used for injection of multiple components in this manner include but are not necessarily limited to those described in U.S. Provisional Patent Application No. 60/878,527 filed Jan. 3, 2007 and in U.S. patent application Ser. No. 11/426,219 filed Jun. 23, 2006 (published as United States Published Patent Application 2007-0014784), which claims priority to U.S. Provisional Patent Application Nos. 60/693,749 filed Jun. 23, 2005 and 60/743,686 filed Mar. 23, 2006, the entire disclosure of each such application being expressly incorporated herein by reference.

The catheter body 13 may also have a guidewire lumen 35 which extends to the distal end 17 of the catheter body 15. In this embodiment, the guidewire lumen 35 extends proximally to an inlet port 37 on the catheter side wall adjacent to the proximal section 23. The catheter body also has a lead lumen 39 for a purpose described below.

In this example, the catheter includes a tapered distal tip section 55 of soft, flexible, biocompatable material and exit port 29 is spaced slightly proximally of shoulder 57.

### Imaging Transducer

An imaging transducer 81 is mounted on the distal tip section 55 just distal to shoulder 57. In this embodiment, the imaging transducer 81 comprises a phased array transducer (e.g., an intravascular ultrasound transducer or IVUS) operative to image 3600 about the catheter 11. This imaging transducer 87 comprises an annular array of individual crystals or elements coupled to a multiplex circuit which is within the major section 51 of the catheter body 13 adjacent the shoulder 57. The multiplex circuit is in turn coupled to leads which extend through the lead lumen 39 and a port or sidearm 83 of the hub 21 to an imaging console. When activated, the imaging transducer 87 emits ultrasound signals and receives back echos or reflections which are representative of the nature of the surrounding environment. The imaging transducer 81 provides an imaging signal from which an image of the surrounding structure can be created by signal processing apparatus located in the imaging console and viewed on a standard display screen. A suitable phased array transducer, the accompanying circuitry and the imaging console may be obtained commercially from Endosonics of Rancho Cordova, Calif. or Intravascular Research Limited (United Kingdom).

### Orientation Marker

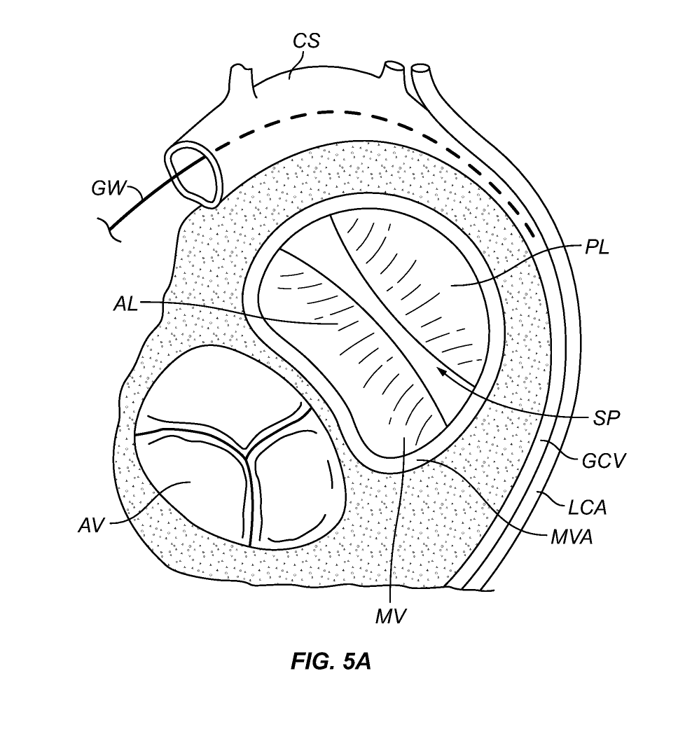

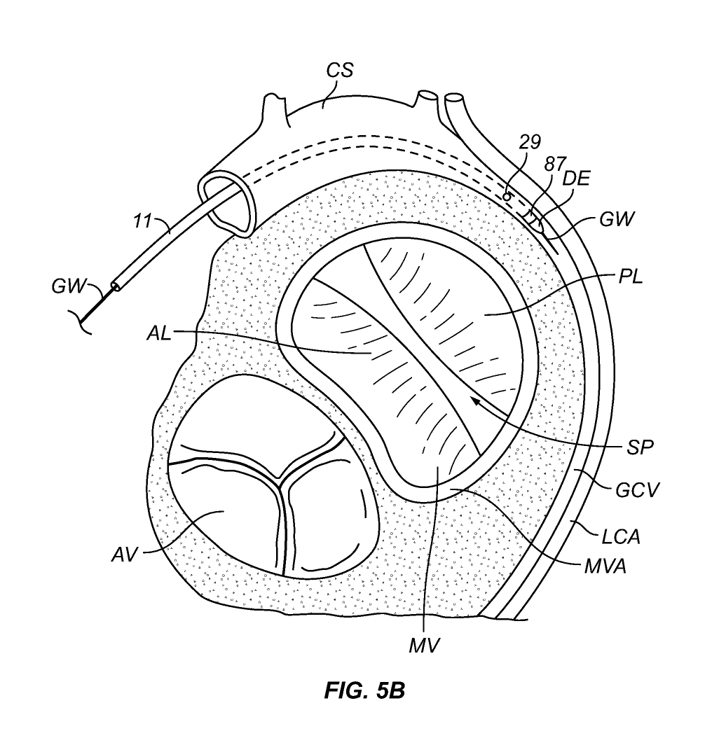

An imageable marker structure 101 is fixedly mounted on the catheter body 13 in a known circumferential orientation relative to the exit port 29. As seen in FIG. 3F, this marker structure 101 is generally in the form cage having three longitudinal members 103 and 103pp. As seen in FIG. 3B, this marker structure 101 is mounted on the catheter such that the transducer 81 is within the longitudinal members 103 and 103pp. The longitudinal members 103 and 103pp are disposed at circumferentially spaced apart locations. Each of these longitudinal members creates a bloom or echo on the ultrasound image, as illustrated in FIGS. 4A and 4B. One of the longitudinal members 103pp is positioned at a circumferential position that is axially aligned with the exit port 29 or otherwise positioned to be indicative of the trajectory on which the tissue penetrator 85 will advance from the catheter body 13 and is designated as the penetrator path indicating member 103pp. As seen on FIGS. 4A and 4B and described more fully herebelow, this penetrator path indicating member 103PP provides a penetrator path indication 147 on the image display, thereby showing the operator a projection of the trajectory that will be followed by the tissue penetrator when the tissue penetrator 85 is subsequently advanced from the catheter body 13.

FIGS. 4A and 4B are an illustration of what the operator may see on the display screen of the imaging console 89 during performance of a method of the present invention using the particular tissue penetrating catheter shown in FIGS. 3A-3G. Specifically, in FIG. 4A, the tissue penetrating catheter 11 has been inserted and advanced to a position within the coronary venous sinus (CVS) or great cardiac vein (GCV). On the image displayed from the imaging transducer 81, one can see the surrounding wall of the coronary venous sinus (CVS) or great cardiac vein (GCV) in which the catheter 11 is positioned as well as an image of the incompetent mitral valve MV. The penetrator trajectory image 147 created by the penetrator path indicating longitudinal member 103pp is visually distinguishable from the images created by the other longitudinal members 103 of the marker structure 101. In the example of FIG. 4A, this penetrator trajectory image 147 is not directed toward the mitral valve MV, but rather is directed to one side of the mitral valve. This indicates that, if the tissue penetrator 85 were to be advanced from the catheter body 13 without first adjusting the rotational orientation of the catheter 11, the penetrator 85 would not travel in the direction of the mitral valve as desired. In view of this, the operator may rotate the catheter 11 until the penetrator trajectory image 147 is directed at the mitral valve MV or otherwise toward the location to which it is intended for the penetrator 85 to advance.

It will be appreciated that, as an alternative to the use of the marker structure 101, the imaging transducer 87 could be mounted in a fixed position and a selected one (or selected ones) of the individual imaging elements (e.g., crystals) of the phased array may be selected as being in longitudinal alignment with the outlet aperture 29 or otherwise located so as to be indicative of the trajectory on which the penetrator 85 will advance from the catheter body 13. This selected imaging element(s) 121 shall be referred to herein as the “penetrator-path-indicating imaging element(s).” The imaging console 86 may include a computer or processor that is programmed to display on the imaging display a marking (e.g., a vertical line or other suitable making) that is in aligned with the radial location of the penetrator-path-indicating imaging element(s). Thus, such marking will serve as a visual indicator of the trajectory that will be followed by the tissue penetrator 85 as it is advanced from the catheter body 13. It will be appreciated by those of skill in the art that this marking may be created on the imaging display screen electronically (e.g., as an illuminated or colored line on the image) or it may be physically marked on the screen (e.g., by felt tipped marker or other suitable marking material or apparatus such as a template). In such embodiments, the operator may rotate the catheter until the marking (e.g., vertical line) passes directly through the image of the cardiac valve to be repaired, thus indicating to the operator that when the tissue penetrator 85 is subsequently advanced from the exit port 29, it will advance toward the cardiac valve annulus and not in some other radial direction.

Also, as an alternative to the use of the marking 101 and any on-board imaging transducer 81, the catheter may include suitable radiographic marking to allow the operator to rotationally adjust and radially orient the catheter using fluoroscopy or other radiographic imaging.

### EXAMPLE 1

### Treatment of Mitral Valve Regurgitation by Injection of a Space Occupying Substance

FIGS. 2A through 2F show steps in a method wherein the above described tissue penetrating catheter device 11 is used to inject a space occupying material at one or more interstitial location(s) within the heart near the mitral valve annulus MVA to cause the posterolateral leaflet PL of the mitral valve MV to move toward the anteromedial leaflet Al, thereby improving the closure of the leaflets and lessening regurgitation through the mitral valve MV.

As seen in FIG. 2A, a guidewire is initially advanced into the coronary sinus CS and, in some cases, may extend into a proximal portion of the great cardiac vein GCV, adjacent to the mitral valve MV. As shown, in this malfunctioning mitral valve MV, a space SP exists between the anteromedial leaflet AL and posterolateral leaflet PL during the systolic phase of the cardiac cycle, when a normally functioning mitral valve would be fully closed.

Thereafter, as shown in FIG. 2B, the tissue penetrating catheter (with its tissue penetrator 85 in the retracted position) is advanced over the guidewire GW to a position where the tissue penetrator outlet port 29 is adjacent to the mitral valve MV. If the catheter 11 is equipped with the optional imaging transducer 87 and orientation structure 101, the imaging transducer will then be actuated and the operator, while viewing an image from the imaging transducer 87, will rotate the catheter 11 as needed until the penetrator path indication 147 is aligned with the interstitial location where it is intended to inject the space occupying material, as illustrated in FIGS. 4A and 4B and discussed above.

After the catheter 11 has been positioned and rotationally oriented so that the penetrator 85 is effectively aimed at the desired location, the penetrator 85 is advanced to the desired location, as seen in FIG. 2C. The advancement and positioning of the penetrator 85 may be monitored or verified using the optional imaging transducer 87 of the catheter 11 and/or other suitable means such as by fluoroscopy.

As seen in FIG. 2D, after is has been determined that the tissue penetration member 85 is at the desired interstitial location, the space occupying material 10a is injected through the penetrating member 85 so that a quantity of that space occupying material 10a accumulates within the myocardium adjacent to the mitral valve annulus MVA. In some applications, quantities of space occupying material 10a may be deposited at multiple locations around the annulus. This exerts pressure on the mitral valve annulus MVA causing some remodeling of the annulus and causing the posterolateral leaflet PL to move toward the anteromedial leaflet AL, thereby reducing or eliminating the space SP that had existed between the leaflets. In some applications, this treatment will actually bring the anterior and posterolateral leaflets AL, PL into normal abutment or coaptation, as shown in the illustration of FIG. 2D. In this manner, the mitral regurgitation will be eliminated or improved. The positioning of the valve leaflets AL, PL may be monitored by echocardiography and/or the competency of the valve may be monitored by dye contrast angiography or other suitable means to determine when the amount of the space occupying material 10a injected has been adequate to bring about the desired improvement in leaflet coaptation or valve function.

Thereafter, as shown in FIG. 2E, the penetration member 85 is again retracted into the catheter 11. Then, as seen in FIG. 2F, the catheter 11 and guidewire GW are removed, leaving the space occupying material 10 in place.

### EXAMPLE 2

### Treatment of Mitral Valve Regurgitation By Implantation of a Space Occupying Device

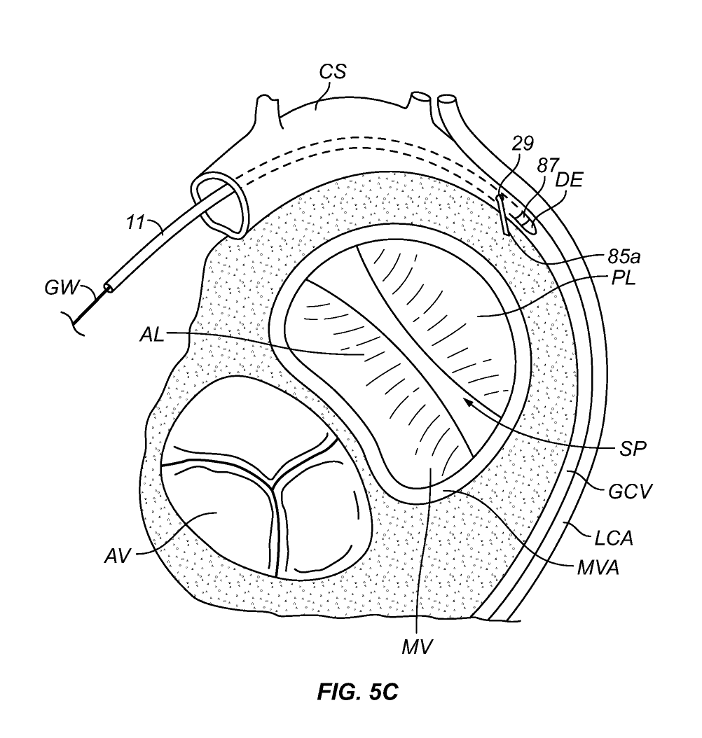

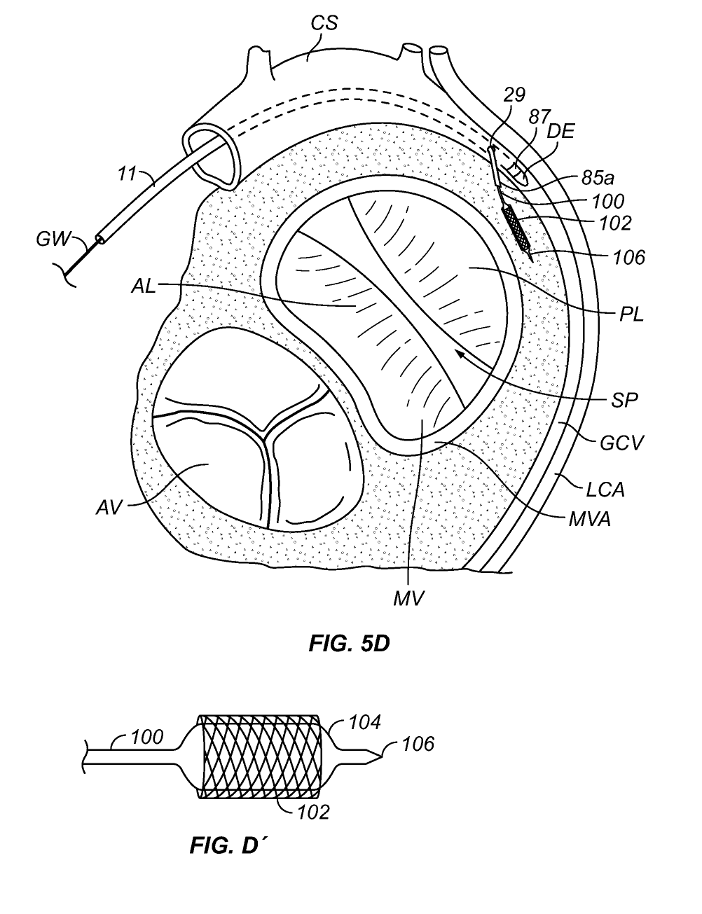

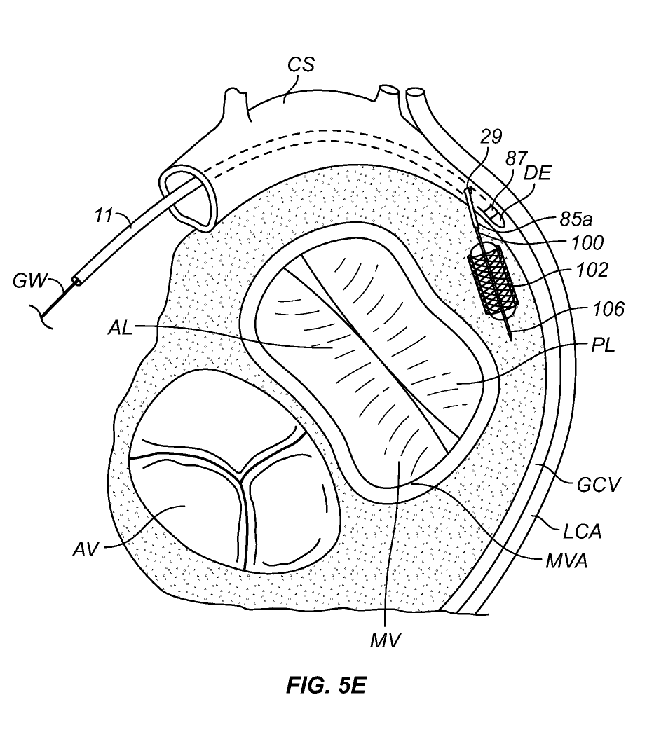

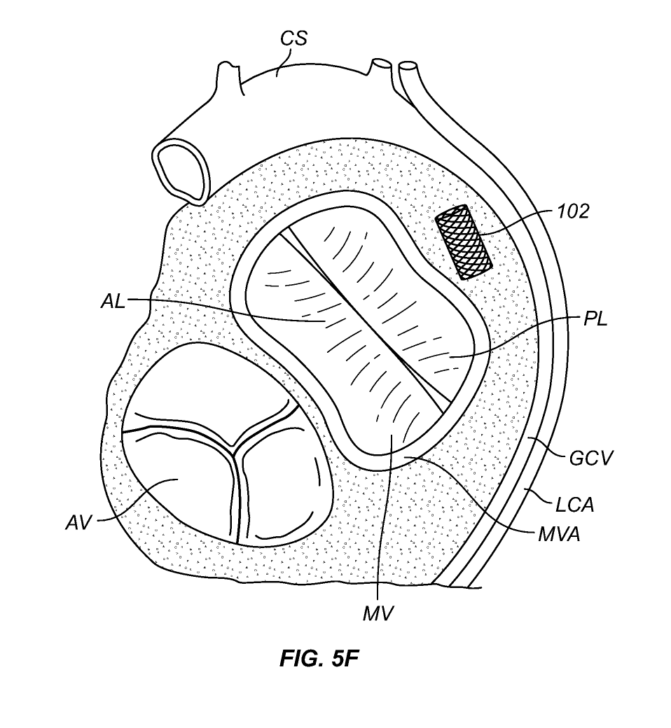

FIGS. 5A through 5F show steps in a method wherein the above described tissue penetrating catheter device 11 is used to implant a space occupying device 10b at one or more interstitial location(s) within the heart near the mitral valve annulus MVA to cause the posterolateral leaflet PL of the mitral valve MV to move toward the anteromedial leaflet Al, thereby improving closure of the leaflets and lessening regurgitation through the mitral valve MV.

As seen in FIG. 5A, a guidewire is initially advanced into the coronary sinus CS and, in some cases, may extend into a proximal portion of the great cardiac vein GCV, adjacent to the malfunctioning mitral valve MV. As shown, a space SP exists between the anteromedial leaflet AL and posterolateral leaflet PL of the valve during the systolic phase of the cardiac cycle, when a normally functioning mitral valve would be fully closed.

Thereafter, as shown in FIG. 5B, the tissue penetrating catheter 11 (with its tissue penetrator 85a in the retracted position) is advanced over the guidewire GW to a position where the tissue penetrator outlet 29 is adjacent to the mitral valve MV. If the catheter 11 is equipped with the optional imaging transducer 87 and orientation structure 101, the imaging transducer will then be actuated and the operator, while viewing an image from the imaging transducer 87, will rotate the catheter 11 as needed until the penetrator path indication 147 is in alignment with the interstitial location where it is intended to inject the space occupying material as illustrated in FIGS. 4A and 4B and described above.

After the catheter 11 has been positioned and rotationally oriented so that the penetrator 85a is effectively aimed at the desired location, the penetrator 85a is advanced into the myocardium to a position where it is adjacent to, and is directed substantially tangential to, the mitral valve annulus MVA. Such advancement and positioning of the penetrator 85a may be monitored or verified using the optional imaging transducer 87 of the catheter 11 and/or other suitable means such as by fluoroscopy.

With reference to FIGS. 5D and 5D′, after it has been determined that the penetrator 85a is in the desired position, a small balloon catheter 100 having a balloon 102 with a generally cylindrical space occupying device 10b mounted thereon is advanced through the penetrator 85a and through some myocardial tissue distal to the penetrator 85a, to a position where the space occupying device 10b is at the location where it is to be implanted. Examples of small balloon-expandable stents that may be used as the space occupying device 10b and delivery catheters therefore include the Guidant MULTI-LINK RX PIXEL® Coronary Stent System (Abbott Vascular, Inc., Santa Clara, Calif.) and the Micro-Driver® Coronary Stent System (Medtronic Vascular, Inc., Santa Rosa, Calif.). Another small balloon catheter device that may be used for delivery and expansion of the space occupying device 10b, such as a balloon-expandable stent, is an occlusion wire having an occlusion balloon with a deflated diameter of about 0.028 inch and a fully inflated diameter of about 5.5 mm (GuardWire® Temporary Occlusion System, Medtronic Vascular, Inc., Santa Rosa, Calif.). The balloon catheter 100 or other delivery catheter used to deliver the space occupying device 10b may in some embodiments have a sharp distal tip 106 to facilitate its desired advancement through tissue.

As seen in FIG. 5E, in this example, the balloon 102 is inflated causing the space occupying device 10b to expand and plastically deform so that it will retain such expanded configuration. Preferably, the device 10b will be positioned relative to the mitral valve MV so that such expansion of the device 10b will cause some remodeling of the mitral valve annulus MVA causing the posterolateral leaflet PL to move toward the anteromedial leaflet AL, thereby eliminating (or in some cases reducing) the space SP. In some applications, this treatment will actually bring the anterior and posterolateral leaflets AL, PL into abutment or coaptation as shown in the illustration of FIG. 2D. This will improve or eliminate the mitral regurgitation. The positioning and or functioning of the leaflets AL, PL may be monitored by echocardiography, contrast angiography or other suitable means to determine when the device 10b has been expanded sufficiently to bring about the desired improvement in leaflet coaptation or valve function. In some other embodiments, the space occupying device 10b may be self-expanding and, as is well known in the art, may be constrained by a sheath, clips, ties or other constraint apparatus during advancement to the implantation site and, hereafter, the constrain apparatus may be removed or deactivated, thereby allowing the space occupying device 10b to expand.

Thereafter, as shown in FIG. 5F, the balloon 102 is deflated, the balloon catheter 100 and penetration member 85 are retracted into the catheter 11 and the catheter 11 and guidewire GW are removed, leaving just the expanded device 10b in place.

It is to be further appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless to do so would render the embodiment or example unsuitable for its intended use. Also, where the steps of a method or process are described, listed or claimed in a particular order, such steps may be performed in any other order unless to do so would render the embodiment or example not novel, obvious to a person of ordinary skill in the relevant art or unsuitable for its intended use. All reasonable additions, deletions, modifications and alterations are to be considered equivalents of the described examples and embodiments and are to be included within the scope of the following claims.

What is claimed is:

1. A method for improving function of an incompetent cardiac valve that has leaflets, said method comprising the step of:
A) implanting a space occupier at an interstitial location(s) within heart tissue such that force exerted by the space occupier causes repositioning of at least one of the valve leaflets to improve competency of the valve.

2. A method according to claim 1 wherein the space occupier comprises a quantity of space occupying substance and wherein Step A comprises delivering said space occupying substance to the interstitial location within heart tissue.

3. A method according to claim 2 wherein the space occupying material is injectable through the lumen of an delivery cannula and wherein Step A comprises:
i) inserting an delivery cannula into the heart; and
ii) injecting the space occupying material through the delivery cannula to form a depot of the space occupying material at the interstitial location within heart tissue.

4. A method according to claim 3 wherein the delivery cannula comprises a needle having one or more lumens.

5. A method according to claim 3 wherein the delivery cannula is advanceable from a tissue penetrating catheter and wherein the step of inserting the delivery cannula into the heart comprises:
inserting the tissue penetrating catheter into the subject's vasculature;
advancing the tissue penetrating catheter through the subject's vasculature to a location within the coronary vasculature;
advancing the delivery cannula from the tissue penetrating catheter and into cardiac tissue; and
injecting the space occupying material through the delivery cannula to form a depot of the space occupying material at the interstitial location within heart tissue.

6. A method according to claim 5 wherein the delivery cannula is part of the tissue penetrating catheter.

7. A method according to claim 6 wherein the tissue penetrating catheter has a tissue penetrator that has a lumen and an open distal end, said tissue penetrator being advanceable from the tissue penetrating catheter into the cardiac tissue, and wherein:
the step of advancing the delivery cannula from the tissue penetrating catheter and into cardiac tissue comprises i) advancing the tissue penetrator from the tissue penetrating catheter into cardiac tissue and ii) advancing the delivery cannula through the lumen of the tissue penetrator and out of its open distal end.

8. A method according claim 7 wherein the delivery cannula comprises a flexible catheter.

9. A method according to claim 8 wherein the delivery cannula has a tissue penetrating distal end so that it may penetrate further through cardiac tissue after exiting the distal end opening of the tissue penetrator.

10. A method according to claim 5 wherein the tissue penetrating catheter is equipped with orientation apparatus useable to determine the trajectory on which the delivery cannula will advance and wherein the method further comprises the steps of:
using the orientation apparatus to determine a projected trajectory on which the delivery cannula will advance; and
adjusting the rotational orientation of the catheter as needed so that the projected trajectory on which the delivery cannula will advance is in the direction of the intended implantation location.

11. A method according to claim 7 wherein the tissue penetrating catheter is equipped with orientation apparatus useable to determine the trajectory on which the tissue penetrator will advance and wherein the method further comprises the steps of:
using the orientation apparatus to determine a projected trajectory on which the tissue penetrator will advance; and
adjusting the rotational orientation of the catheter as needed so that the projected trajectory on which the tissue penetrator will advance is in the direction of the intended implantation location.

12. A method according to claim 5 wherein the tissue penetrating catheter is inserted into the subject's venous vasculature and advanced into the coronary sinus or a coronary vein of the subject's heart.

13. A method according to claim 5 wherein the tissue penetrating catheter is inserted into the subject's arterial vasculature and advanced into a coronary artery.

14. A method according to claim 2 wherein the space occupying substance is selected from the group consisting of:
A) collagens;
B) hyaluronic acid;
C) polymeric materials; and
D) hydrogels.

15. A method according to claim 2 wherein the space occupying substance expands after it has been delivered to the interstitial location within heart tissue.

16. A method according to claim 1 wherein the space occupier comprises a space occupying device.

17. A method according to claim 16 wherein the space occupying device is selected from the group consisting of beads, balls, filaments, stents, cages, expandable structures, implantable balloons, implantable balloons filled with solid or gellatenous material and implantable tissue expanders.

18. A method according to claim 16 wherein the space occupying device is expandable from a non-expanded configuration to an expanded configuration and wherein the method comprises:
i) advancing the space occupying device to the interstitial location within heart tissue while in its non-expanded configuration; and
ii) causing the space occupying device to expand to its expanded configuration.

19. A method according to claim 18 wherein the space occupying device self-expands and wherein constraint is applied to the space occupying device so that it is constrained in a non-expanded configuration while being delivered to the implantation location and, thereafter, the constraint is removed to allow the space occupying device to self-expand to an expanded configuration.

20. A method according to claim 18 wherein the space occupying device is plastically deformable to its expanded configuration and wherein the space occupying device is delivered to the interstitial location within heart tissue while in its non-expanded configuration and is thereafter plastically deformed to its expanded configuration.

21. A method according to claim 20 wherein the space occupying device is delivered by a delivery catheter that has a balloon, and wherein, during delivery of the space occupying device to the implantation location, the balloon is deflated and the space occupying device is mounted on the deflated balloon and, thereafter, the balloon is inflated thereby causing the space occupying device to expand to its non-expanded configuration.
